(19) United States
(12) Patent Application Publication
(10) Pub. No.: US 2004/0013563 A1
(43) Pub. Date: Jan. 22, 2004

(54) Cooling tower maintenance

(76) Inventors: Edward Adolph Romer, Killara (AU); Robert Edward Romer, Mona Vale (AU); Geoffrey John Daly, Gordon (AU)

Correspondence Address: Mark D Wieczorek, PO Box 70072, San Diego, CA, 92167, US

(21) Appl. No.: 10/276,583

(22) PCT Filed: Apr. 26, 2001
(86) PCT No.: PCT/AU01/00469

(30) Foreign Application Priority Data
May 19, 2000 (AU) PQ 7613
Jun. 16, 2000 (AU) PQ 8192

(51) Int. Cl. A01N 1/00; A61L 2/00; A61L 9/00

(52) U.S. Cl. 422/28; 422/37; 261/DIG. 11

(57) ABSTRACT

The present invention discloses the use of an electrolysis cell (30,40,50-52) to provide for super chlorination of cooling tower water. Halogens naturally present in the water are concentrated by evaporation and provide the starting material for the production of oxyhalite compounds (typically to a level of superchlorination) as a biocide. The biocide is recycled thereby avoiding the cost of chemicals. The level of atmospheric polluants, and particulate matter especially, is thought to be the determining factor in biocide consumption.

### FIELD OF THE INVENTION

The present invention relates to cooling towers, decorative fountains, and like structures having a body of circulating water. The circulating water is home to microbial life forms or micro-organisms (including the bacterium which produces Legionnaires disease) growing in, and colonising the water. In order to prevent such colonisation biocides in the form of chemicals are added at regular intervals to the water to kill or treat the microbial life forms. The present invention is concerned with increasing the efficiency and efficacy of the biocide activity.

### BACKGROUND ART

Such cooling towers are well known in industrial equipment especially in relation to air-conditioning equipment and providing chilled water to a wide variety of heat generating industrial equipment including, for example, injection moulding machines, electric power stations and to a wide variety of industries including the food processing industry and the automotive washing industry. The present invention also finds application in the production of sterile water such as may be used in dental surgeries and aged care facilities, in the poultry industry to combat Newcastle disease, in the horticultural industries to combat algal and fungal growths, etc. Evaporation air cooling towers are used to regulate the heat transfer involved in maintaining relatively constant air and water temperatures.

Because the chilling of the water is carried out by means of evaporation, in a substantial counter airflow, the circulating water is liable to create small aerosol size particles of water which become entrained in the airflow. These water aerosols can move some distance from the premises where they were generated. Such small water particles are able to be breathed into the lungs of passers-by and in this way Legionnaires disease can be inadvertently contracted by the general public. For this reason there is considerable regulatory interest in the ensuring that such pieces of equipment are free from microbial infestation.

Furthermore, such pieces of equipment include heat exchangers and the like having metal parts made from steel and copper which are subject to corrosion from the chemicals used as the biocide. Thus care must be taken to ensure that the biocide is effective against the microbial life forms but does not adversely affect the operating life of the metal parts due to increased corrosion.

The aim of the present invention is to overcome, or at least ameliorate, some of the disadvantages of the prior art in this area and to improve aspects of the performance of such equipment.

### SUMMARY OF THE INVENTION

According to a first aspect of the present invention there is disclosed a method of operating a cooling tower or like structure having a body of recirculating water, air/water interface means to bring the recirculating water into contact with the air, and biocide means to kill or treat microbial life forms in the recirculating water, said method comprising the step of increasing the activity of said biocide means in response to increases in particulate pollutants in said air.

In accordance with the second aspect of the present invention there is disclosed a cooling tower or like structure having a body of recirculating water, air/water interface means to bring the recirculating water into contact with the air, biocide means to kill or treat microbial life forms in the circulating water and means to increase the activity of said biocide means in response to increases in particulate pollutants in said air.

In accordance with the third aspect of the present invention there is disclosed a cooling tower or like structure in which water is recirculated and having a pond to retain said water, and pump means to move said water from said pond through a heat source to heat said water and thence to an air/water interface means comprising at least one spray nozzle distributing a spray of water onto fill material in the presence of a counter flow of air whereby said water is cooled by partial evaporation thereof, said air/water interface means being located upstream of said pond, and wherein the recirculation circuit for said water includes at least one electrolytic cell.

In accordance with the fourth aspect of the present invention there is disclosed a method of operating a cooling tower or like structure in which water is recirculated from a pond to retain said water, by pump means which moves said water from said pond through a heat source to heat said water and thence to an air/water interface means having at least one spray nozzle directing a spray of water onto fill material in the presence of a counter flow of air whereby said water is cooled by partial evaporation thereof and after exiting said air/water interface means returns to said pond, said method including the step of including at least one electrolytic cell in the recirculation circuit for said water.

In accordance with the fifth aspect of the present invention there is disclosed a method of operating a cooling tower or like structure having a body of recirculating water, air/water interface means to bring the recirculating water into contact with the air, and biocide means to kill or treat microbial life forms in the recirculating water, said method comprising the step of substantially recycling chemical components of said biocide means.

In accordance with the sixth aspect of the present invention there is disclosed a cooling tower or like structure having a body of recirculating water, air/water interface means to bring the recirculating water into contact with the air, biocide means to kill or treat microbial life forms in the recirculating water, and recycling means to recycle chemical components of said biocycide means.

In accordance with the seventh aspect of the present invention there is disclosed a method of reducing corrosion in cooling towers or like structures having a body of recirculating water, air/water interface means to bring the recirculating water into contact with the air, and biocide means to kill or treat microbial life forms in the recirculating water and generate oxyhalogen compounds through the operation thereof, said method comprising the step of converting at least some of oxyhalogen compounds into free halogen.

In accordance with the eighth aspect of the present invention there is disclosed apparatus for reducing corrosion in cooling towers or like structures having a body of recirculating water, air/water interface means to bring the recirculating water into contact with the air and biocide means to kill or treat microbial life forms in the recirculating water and generate oxyhalogen compounds through the operation thereof, said apparatus comprising converter means to convert at least some of said oxyhalogen compounds into free halogen

In accordance with the ninth aspect of the present invention there is disclosed a method of maintaining the operation of a cooling tower or like structure substantially free from microbial contamination, said cooling tower comprising a body of recirculating water and air/water interface means to bring the circulating water into contact with the air, said method comprising the step of maintaining said water in a superhalogenated equilibrium state by the continuous production of oxyhalogen compounds.

In accordance with the tenth aspect of the present invention there is disclosed apparatus for maintaining the operation of a cooling tower or like structure substantially free from microbial contamination, said cooling tower comprising a body of recirculating water and air/water interface means to bring the recirculating water into contact with the air, said apparatus comprising a biocide means in fluid communication with said water and operable to maintain said water in a superhalogenated equilibrium state by the continuous production of oxyhalogen compounds.

In accordance with the eleventh aspect of the present invention there is disclosed a method of closing down the operation of a cooling tower or like structure to permit resumption of said operation within a short period, said cooling tower comprising a body of recirculating water, air/water interface means to bring the circulating water into contact with the air, and biocide means to kill or treat microbial life forms in the recirculating water, said method comprising the steps of achieving a equilibrium state of superhalogenation in said circulating water prior to closing down, and maintaining said equilibrium state of superhalogenation in said water after shut down, said state of superhalogenation being achieved and maintained by the production of oxyhalogen compounds from said water.

In accordance with the twelfth aspect of the present invention there is disclosed apparatus for closing down the operation of a cooling tower or like structure to permit resumption of said operation within a short period, said cooling tower comprising a body of recirculating water, air/water interface means to bring the recirculating water into contact with the air, and biocide means to kill or treat microbial life forms in the recirculating water, said apparatus comprising first halogenation means to achieve a equilibrium state of superhalogenation in said water prior to closing down, and second halogenation means to maintain said equilibrium state of superhalogen in said water after shut down, said first and second halogenation means being operable to produce oxyhalogen compounds from said water.

In accordance with the thirteenth aspect of the present invention there is disclosed a method of a method of increasing the sterility of gas by bringing same into contact with superhalogenated water, said method comprising the step of superhalogenating said water by electrolysis

In accordance with the fourteenth aspect of the present invention there is disclosed an apparatus for increasing the sterility of gas, said apparatus comprising first means to bring said gas into contact with water, and electrolysis means to superhalogenate said water.

### BRIEF DESCRIPTION OF THE DRAWINGS

Embodiments to the present invention will now be described with reference to the drawings in which:

FIG. 1 is a schematic diagram illustrating the operation of a conventional cooling tower,

FIG. 2 is a similar diagram but illustrating the operation of a cooling tower in accordance with the first embodiment of the present invention,

FIG. 3 is a view similar to FIG. 2 but of the second embodiment of the present invention,

FIG. 4 is a view similar to FIGS. 2 and 3 but of a third embodiment of the present invention.

FIG. 5 is a schematic circuit diagram of an electrolysis cell controller, and

FIG. 6 is a schematic circuit diagram of a neighbourhood cell control system.

### DETAILED DESCRIPTION

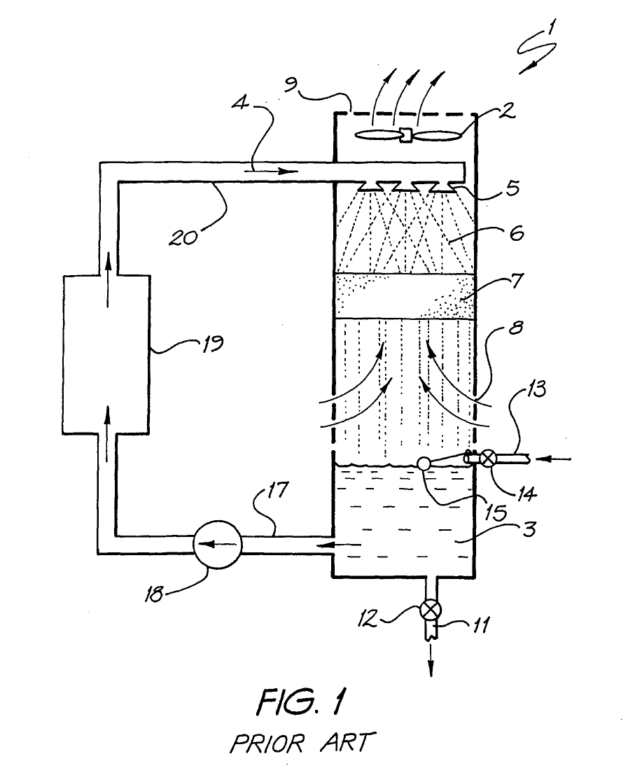

The prior art cooling tower 1 illustrated in FIG. 1 is a generally vertically arranged structure having at least one extractor fan 2 adjacent its top and a pond 3 which acts as a reservoir for circulating water 4, adjacent its base. Positioned below the fan 2 are a number of nozzles 5 which direct downwardly and distribute a spray 6 of the circulating water 4 onto fill material 7. The fill material 7 typically takes the form of beads, hollow tubes, or similar such particles which have a very large surface area in proportion to their volume. A number of air inlets 8 are provided between the fill material 7 and the pond 3 and air outlets 9 are positioned at the top of the cooling tower 2 above the extractor fan 2.

The pond 3 is provided with a drain 11 which is closed by a cock 12. The pond 3 is also provided with an inlet 13 which is connected to the mains water supply and controlled by a valve 14 having a float 15.

Chilled water leaves the pond 3 via an outlet pipe 17 which includes a pump 18 to circulate the water 4 via a heat exchanger 19 and an inlet pipe 20 which is connected to the nozzles 5.

In operation, the circulating water 4 is pumped from the pond 3 by the pump 18 through the heat exchanger 19 where it is heated and returned through the inlet pipe 20 to the cooling tower 1. The water there is ejected through the nozzles 5 where it is formed into a fine spray 6 which is directed downwardly onto the fill material 7, the large surface area of which is thereby wetted. A counter-directed airflow indicated by the arrows in FIG. 1 is caused by the extractor fan 2 and causes air to flow over the fill material 7 and through the spray 6 before exiting via the air outlets 9. This flow of air causes evaporation of the circulating water 4 thereby removing latent heat and chilling the circulating water 4. Also carried out the air outlets 9 are small aerosol size particles of water which, if they contain bacteria such as that which can cause Legionnaires disease, can represent a substantial health hazard.

In order to provide the necessary biocidal action to kill microbial life forms which would otherwise generate within the circulating water 4, various chemical additives such as liquid chlorine, hypochlorite or chlorinated phenols which are oxidizing biocides, or non-oxidizing biocides such as isotriazoline are added to the water. Such chemical biocides have numerous problems. For example, isotriazoline must be handled carefully since it can produce dermatitis on contact with the skin. It is also an antibiotic and therefore needs to be used sparingly in order to prevent resistant mutations of the microbial life forms it is intended to kill, from arising. Further such chemicals must be manually handled, particularly up ladders and/or in confined spaces.

Irrespective of whether oxidizing biocides or non-oxidizing biocides are used, halite or oxyhalite ions and particularly oxychlorite ions, will be produced. In the case of oxidizing biocides these are normally produced directly, however, in the case of non-oxidizing biocides these are produced as metabolic by-products. The end metabolic product from the use of oxydising and non-oxydising biocides is the choride or halide ion.

Not only are such halite and halide ions corrosive in their own right, but they also create a galvanic couple which can lead to rampant galvanic corrosion. For this reason corrosion inhibitors are generally also added to the circulating water 4. This adds to the total expense of added chemicals. Despite the existence of the corrosion inhibitors, it is not unknown for the metallic parts of the circulating water circuit to be corroded within two years if the addition of chemicals is not done skillfully. A further factor in this connection is that the circulating water is maintained in intimate contact with large volumes of air and is therefore saturated in relation to dissolved oxygen. Since corrosion requires oxygen this dissolved oxygen accelerates the rate of corrosion.

Traditionally, the circulating water is intended to be maintained at a free chlorine level of approximately 0.5 ppm. This intended concentration is not increased for two reasons, firstly because of fear of corrosion and secondly, the cost of the chemicals required to increase the free chlorine concentration above 0.5 ppm is regarded as being too expensive.

The free chlorine level (or available chlorine level) refers to the available concentration of various chlorine compounds or ions such as hypochlorous acid (HOCl), monochloramine (NOCl), sodium hypochlorite (NaHOCl) or chlorine dioxide (ClO₂). The test which determines whether or not available or free chlorine is present is the ability to react with potassium iodide (KI) in acid solution to release free iodine (I₂). The percentage amount of free or available chlorine is obtained by comparing the amount of iodine liberated from the same weight of chlorine. When chlorine reacts with potassium iodide under the above conditions, each gram of chlorine liberates 3.6 grams of iodine. Thus for a chlorine compound it is necessary only to calculate the amount of iodine liberated by 1 gram of the chlorine compound under the above conditions. This amount divided by 3.6 gives the free or available chlorine. This can be expressed as a percentage by multiplying by 100 or as a concentration by expressing the amount of iodine liberated divided by 3.6 as parts per million.

The cooling towers 1, in some jurisdictions are required to undergo an expensive maintenance routine. For example, in the Australian state of New South Wales once a month chemicals are added to take the free chlorine concentration up to 5 ppm above the normal maintenance dose (that is to 5.5 ppm) for half an hour to one hour. This time is limited because of fear of corrosion of the system. After this limited time the cooling tower operation is stopped, the water in the pond 3 is drained via the drain 11 and the entire system flushed with fresh water. The flushing water is then drained, the pond 3 re-filled, chemicals added to bring the free chlorine concentration up to 0.5 ppm and the cooling tower is then returned to operation.

In the event of a shut down for a number of working days, for example over the Christmas/New Year period or the Easter holidays, or because of maintenance work on the heat exchanger 19 (or similar equipment) then a shut down routine is followed. This involves increasing the free chlorine concentration to 25 ppm for approximately ½ hour. Then the water 4 is drained from the pond 3 and the system flushed. Then the system is re-filled and chemically dosed to a free chlorine concentration of 10 ppm. This water is then circulated for 1 hour and then drained.

On re-starting the equipment the pond 3 is again filled with water which is dosed to a free chlorine concentration of 25 ppm, and then circulated for 1/2 hour before being drained and flushed. Then the pond 3 is again refilled, dosed to 10 ppm, the water circulated for 1 hour and then drained. Finally, the pond 3 is again re-filled, dosed to the standard 0.5 ppm free chlorine concentration and the industrial process is then able to resume. This is an expensive procedure often taking 2 men 1 day and using considerable amounts of expensive chemicals.

Similarly, in the event of an outbreak of Legionnaires disease, then an emergency routine is put in place. Typically this involves increasing the free chlorine ion concentration to 25 ppm by adding liquid chlorine in some form, typically hypochlorite. This elevated level, which constitutes super chlorination, is only maintained for half an hour because it is thought to cause substantial corrosion because of the high chloride level (17%) in the hypochlorite solution. At the end of the half hour period the pond 3 is drained and the system flushed. Then the system is recharged with water and chemicals added to bring the free chlorine concentration up to 10 ppm for a period of one hour. Again this period is limited because of corrosion fears. The system is then again drained and flushed and recharged with fresh water to which chemicals are added so as to raise the free chlorine concentration level to 0.5 ppm and the maintenance schedule is then resumed.

In a typical cooling tower 1, the volume of the circulating water is approximately 1,500 litres and the flow generated by the pump 18 is approximately 25 cubic metres/hour. Approximately 3% of the circulating water 4 is lost as a result of evaporation and a further 0.2% is lost as a consequence of drift of small water droplets. In order to prevent a build up of slag within the circulating water 4 the circulating water 4 is bled by being drained out the drain 11 from time to time and the bleed loss is approximately 0.8% of the circulating volume. In order to maintain the volume of the circulating water 4, make-up water is supplied via the inlet 13 and the make-up volume is approximately ten times that bled out through the drain 11.

It is known to provide a sensor probe to attempt to measure the free chlorine concentration in the circulating water 4. Such probes, known as “redox units”, utilize a current flowing in the circulating water 4 between two adjacent electric terminals immersed in the water 4. However, calcium deposits quickly build up on the terminals and this renders the readings inaccurate. Indeed, the probe readings are more a measure of the probe cleanliness than the free chlorine concentration. For this reason such probes have remained as laboratory instruments rather than standard industry sensors.

The prior art system described generally in FIG. 1 suffers from a number of very serious disadvantages. In particular, the efficacy of the biocide activity is not guaranteed and despite the high expense of the regular maintenance, from time to time outbreaks of Legionnaires disease occur, often with fatal result. In addition, corrosion of the industrial equipment being cooled is a serious problem. Finally, the expense of the maintenance and chemicals required for the biocide activity is very substantial and cooling towers are known for being expensive items of equipment to maintain and operate.

Similar considerations apply in other equipment where there is a body of circulating water and the circulating water is in contact with the air, particularly where large areas of wetted surface are in contact with the air. Examples include decorative fountains where a spray of water is produced which travels through the air. Other examples include motor vehicle washing installations where the washing water is recycled after flowing as a thin sheet over substantial areas of floor, and air scrubbers.

The present invention arises because of a desire by the inventors to utilise electrolysis as a means of creating the necessary biocidal action. In particular, in the swimming pool industry it is well known to add a halogen salt, such as sodium chloride, to the slightly alkaline (eg pH of from 6.9 to 8.0) swimming pool water in order to provide a source of halide ions (e.g. chloride ions) in the water. Then a conventional electrolysis cell can be used to form halogen gas (e.g. chlorine gas) which has a biocidal action as the bubbles of chlorine gas generated are dissolved back into the water. However, in most swimming pool installations, the pipes are fabricated from plastics material and therefore corrosion is not an issue.

However, in cooling tower operations with the supply of chilled water, in order to provide an adequate heat exchange, metallic pipes must be used because plastics material is normally a thermal insulator. Further the severe corrosion problems inherent with such cooling tower operations, which have been described above in relation to FIG. 1, means that adding salt to the circulating water would represent a retrograde step. U.S. Pat. No. 4,790,923 (Stillman) is indicative of the swimming pool art and teaches the adding of halogen salts, such as sodium chloride, to the water to be the subject of the biocidal action, in order to provide sufficient chloride ions to provide an effective level of electrolysis. For example, Stillman discloses sodium chloride concentrations of, for example, 1 g/l which constitutes 1,000 ppm and results in free chlorine levels of the order of 1-2 ppm. Since super chlorination is generally regarded as free chlorine levels of approximately 6-10 ppm or higher, this prior art is not operating in a superchlorinated state.

In U.S. Pat. No. 5,439,576 (Schoederl) an electrolysis cell is disclosed which it is claimed enables water to be sterilized without adding chlorine compounds. It is said that fresh water, which includes low chloride concentrations under 10 mg/l (which corresponds to 10 ppm) can be sterilized utilising the cell. Typical cell efficiencies are approximately 20% and thus this indicates that the cell of this specification results in a free chlorine concentration of approximately 2 ppm.

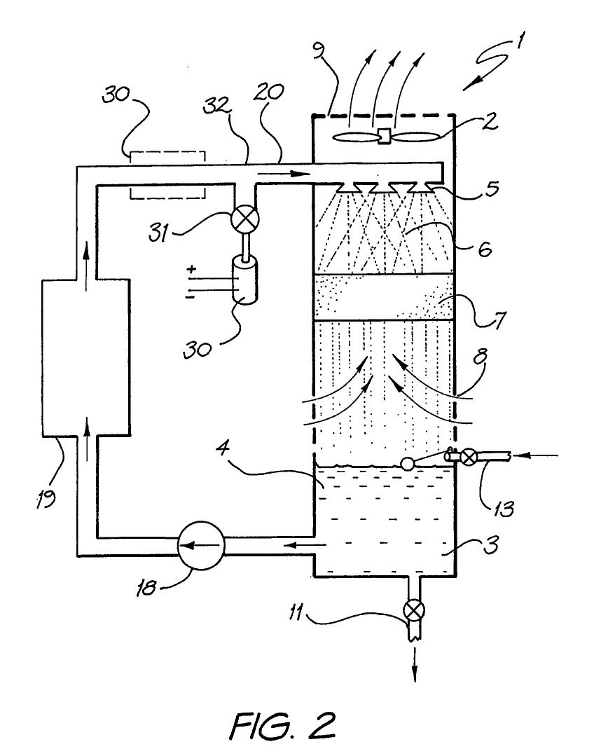

Turning now to FIG. 2, illustrated therein is an experimental apparatus manufactured by the inventors which is substantially identical to the prior art cooling tower of FIG. 1 and like numbers are used to designate like parts. The difference is that an electrolysis cell 30 is connected via a valve 31 and a tee-junction 32 into the inlet pipe 20 immediately upstream of the nozzles 5. The cell 30 could have been connected “in line” as indicated by broken lines in FIG. 2. However, this configuration was not adopted since the cell produces calcium deposits and therefore requires regular cleaning. Also such deposits may block the nozzles 5. The valve 31 enables the cell 30 to be isolated from the circulating water 4 and cleaned as necessary.

The electrolysis cell 30 produces bubbles of gas. As these bubbles rise under the buoyancy force experienced by the bubble, the upward motion of the bubble entrains water to flow upwardly through the valve 31 and into the inlet pipe 20. Simultaneously, some water also flows downwardly into the cell 30 from the pipe 20 in order to replace the upwardly moving water. Thus water flows in opposite directions within the cell 30.

The cooling tower water is generally alkaline in nature having a pH greater than 7, typically approximately 8-8.5. Under these conditions it is thought that several general reactions apply. These are as follows:—

NaCl+H₂O→NaOCl+H₂ (gas) (1)

NaOCl+H₂O⇄NaOCl+HOCl (2)

2Cl⁻+2H₂O→2HOCl+H₂ (gas)+2e (3)

Reactions 1 and 3 proceed only from left to right and result in the liberation of hydrogen gas which is safely entrained in the air flow through the cooling tower 1 and vented to the atmosphere. Reaction 2 is reversable and the percentage of HOCl relative to NaOCl depends on the pH of the solution, with increasing pH resulting in decreasing percentage of HOCl. It is the HOCl which is the biocide and it is consumed in the killing of bacteria, etc.

Although the above equations are expressed in terms of sodium and chlorine, they are equally applicable to potassium, for example, and other halogens. Thus they can be expressed in the more general form

AZ+H₂O→AOZ+H₂ (gas) (4)

AOZ+H₂O⇄AOH+HOZ (5)

2Z⁻+2H₂O→2HOZ+H₂ (gas)+2e (6)

where A is Na, K, etc. and Z is F, Cl, Br or I.

The important principle is that chlorine (and other halogens) naturally present in the cooling tower water is converted by the electrolytic cell 30 into oxychlorine (or any halite) compounds which give rise to free or available chlorine and the biocidal action. Furthermore, the halogen is recycled in the process and thus does not need to be continually added.

Although the above concentrates on hypochlorous acid (HOCl) and the hypochlorite ion (OCl⁻), other oxyhalite compounds/ions are also present including perchlorates (ClO₄⁻), chlorates (ClO₃⁻), chlorine dioxide (ClO₂⁻) and monochloroamine (NOCl⁻). Other halogen equivalent members of such compounds including HOF etc. can also be present.

Most city drinking water contains a small level of dissolved chlorine in the form of chloride ions and, in addition, most city water supplies are fluoridated. For the major cities of Australia the following table applies:

| | Chloride ion concentration | |
|---|---|---|
| Adelaide | 60-280 | ppm |
| Brisbane | 30-130 | ppm |
| Canberra | 5-10 | ppm |
| Darwin | 5-10 | ppm |
| Hobart | 5-10 | ppm |
| Melbourne | 5-15 | ppm |
| Perth | 70-260 | ppm |
| Sydney | 20-30 | ppm |

Although the drinking water is fluoridated, the fluorine is mainly present as a silicate compound (e.g. NaFSiO₂) which is relatively unreactive. The fluoride concentration is typically less than 1 ppm.

Based on the above levels of chloride ions available in the mains water supply to the cooling tower 1 of FIG. 2, the inventors anticipated that the level of free chlorine in the circulating water 4 would be approximately 2-20 ppm or about 20% of the chloride level. However, upon measuring the level they found a much higher level than anticipated. As a consequence, the inventors postulate that the action of the cooling tower is to increase the concentration of dissolved matter in the circulating water 4. Since the circulating water 4 is continually losing water as a result of evaporation, and because the dissolved matter does not evaporate, the concentration of dissolved matter increases as the evaporation continues, notwithstanding the addition of make-up water via inlet 13 and the removal of bleed water via a drain 11. It has been found experimentally that the concentration by a factor of approximately 10 of such dissolved matter is the steady state result.

Furthermore, although the load of the heat exchanger 19 was substantially steady and the cooling tower 1 of FIG. 2 was operating at a substantially uniform rate with a substantially uniform load, there were inexplicable changes in the measured value of the free chlorine concentrations in the circulating water 4. After a lengthy and exasperating search for the cause of such variations, the inventors discovered that this was brought about by changing levels of atmospheric pollutants present in the air counterflowing through the cooling tower 1.

Atmospheric pollutants are conveniently divided into four categories. The first is the concentration of ozone in the air. The second is the concentration of various oxides of nitrogen (NOx). The third is the concentration of reactive organic compounds (ROC). The fourth is the concentration of particles or particulate matter. This is thought to be largely, but not completely, determined by the concentration of ROC's. Dust also contributes, however. It is possible to measure by laser beam scattering the levels of particulate matter in the air and two convenient references chosen in such measurements are respectively PM10 meaning the concentration of particulate matter in the air where the particles have a diameter of less than or equal to 10 micrometres, and PM2.5 where the concentration is of those particles having a diameter of less than or equal to 2.5 micrometres.

The inventors have empirically determined that the correlation between change in free chlorine concentration and the change in pollution levels is largely due to changes in the level of particulate matter in the air. Thus a dusty or polluted atmosphere may have a measured PM10 value approaching 100 microgrammes per cubic metre. A typical low level of pollution would have a PM10 value of approximately 10.

The inventors have empirically determined that approximately 9 grams of free chlorine in the circulating water is required to oxidise 1 gram of PM10 particulate matter which becomes adsorbed on, or absorbed into, the circulating water 4 as a result of the action of the air/water interface created by the spray 6, fill material 7 and counter flowing air through the cooling tower 1. Typically the air flow through the cooling tower is of the order of 15,000 cubic metres per hour.

A typical high level of PM10 particulate matter is 90 microgrammes per cubic metre and this when multiplied by the above airflow gives a particulate matter of 1.35 grams per hour which is being delivered by the airflow through the cooling tower 1. Since approximately 9 grams of free chlorine are required to oxidize this particulate matter, this means that the demand for free chlorine is approximately 12.15 grams per hour.

However, on low pollution days the demand for free chlorine is only one ninth this level, or 1.35 grams per hour.

The cell 30 was producing approximately 11 grams per hour of free chlorine since for each Amp at 8 volts Coulomb's Law predicts that one gram per hour of free chlorine is produced and the cell 30 was drawing 11 amps at 8 volts.

As a consequence of these numerical relationships, whilst on days of low pollution there was more than an adequate level of free chlorine within the circulating water 4 to ensure an effective biocidal action, on days of high pollution, the pollutants carried into the cooling tower and its circulating water via the counterflowing air meant that the free chlorine was exhausted. During such times the microbial level within the circulating water 4 can increase rapidly. This observation also offers an explanation as to why outbreaks of Legionnaires disease, which can result from increased microbial levels within the circulating water 4, seem to incur inexplicitably without any rhyme or reason.

The inventors have therefore discovered that by maintaining the circulating water 4 in a superchlorinated (or superhalogenated) condition so as to give a free chlorine (or halogen) level of approximately 15-20 ppm in times of low pollution such as autumn, this provides a safe buffer during times of high pollution where the level of pollutants and PM10 particulates, in particular, reduces the free chlorine level to as low as 2-3 ppm. However, this is still a safe level and therefore indicates that the level of 15-20 ppm provides a safe buffer. High pollution levels can occur at any time—especially if demolition activity commences at the building next door—but are more often encountered in spring because of high pollen levels.

It will also be appreciated by those skilled in the art, that once the relationship between pollutants and biocide consumption is grasped, it is possible to regulate the chemical consumption of prior art devices, such as that of FIG. 1, in order to ensure continuous safe operation.

Experimental results to date indicate that a cooling tower 10 operated over several months in accordance with the arrangement of FIG. 2 maintained the circulating water 4 clear with no growth of algae and no appreciable corrosion. Furthermore, mild steel and copper corrosion coupons immersed in the circulating water 4 gave corrosion rates of 0.5323 thousands of an inch per year (0.0135 mm/year) and 0.0220 thousands of an inch per year (0.0006 mm/year) respectively. Typical minimum acceptable standards for corrosion of mild steel and copper are 6 and 0.5 thousands of an inch per year respectively. So the mild steel corrosion is acceptable and the copper corrosion virtually negligible.

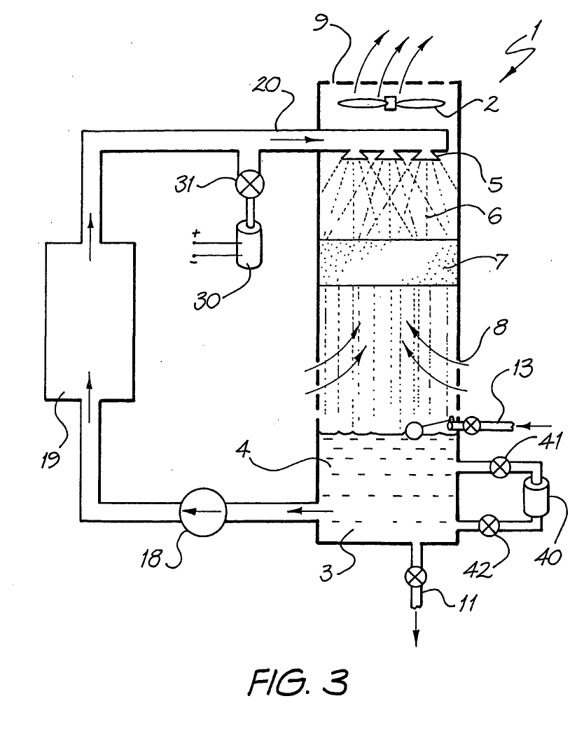

Turning now to FIG. 3, in a second embodiment of the present invention a second electrolysis cell 40 can be connected to the pond 3 via stop cocks 41 and 42 as illustrated. The stop cocks 41 and 42 enable the cell 40 to be isolated from the pond 3 for the purposes of cleaning. As before the generation of gas within the cell 40 causes an upward water flow or motion through the cell 40 and therefore a circulation of the water 4 between the pond 3 and the cell 40 due to the electrolysis action itself.

The additional cell 40 provides a particularly useful function in the event of factory shutdown. With the conventional arrangement illustrated in FIG. 1, in order to stop the cooling tower 1 for several days, such as occurs on holiday periods over Easter, Christmas/New Year and the like, it is necessary to chemically superchlorinate the circulating water 4 and then drain the pond 3 of all the water 4 as explained above. Prior to restarting the cooling tower 1 the pond must be refilled and the water chemically super chlorinated so as to restart the maintenance regime. This is both time consuming and expensive.

However, with the embodiment illustrated in FIG. 3 the electrolysis effect of the additional cell 40 is sufficient to maintain all the water in the pond 3 with the cooling tower stopped in a superchlorinated condition and therefore effective biocidal action is guaranteed. Furthermore, the superchlorinated water can then be repumped through the pipes 17, 20 on re-starting of the industrial process to which the chilled water is delivered with the guaranteed knowledge that even if there might be some microbial growth within the heat exchanger 19, for example, then the superchlorinated water will effectively kill all such microbes rapidly, before any have a chance to escape via the air outlets 9.

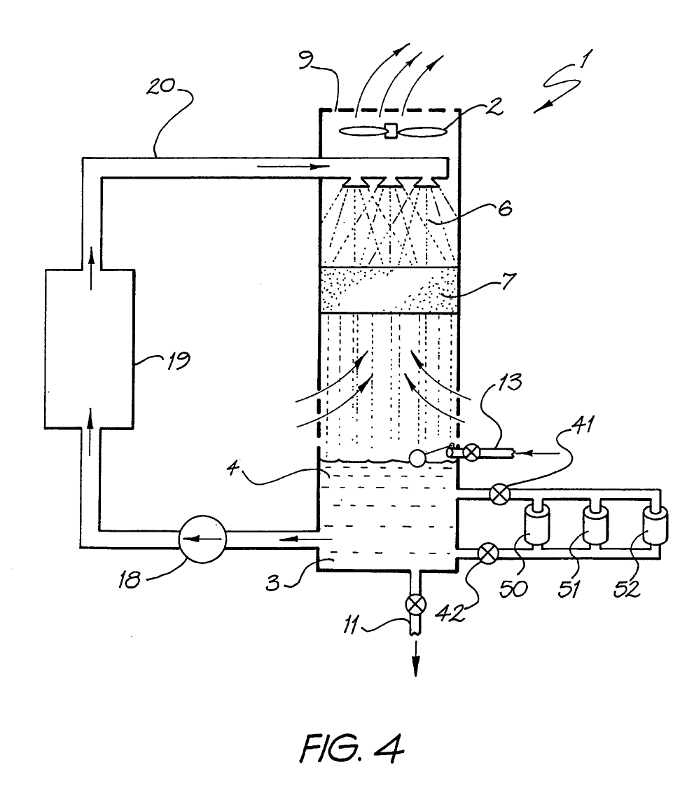

Turning now to FIG. 4 here a further embodiment of the present invention is illustrated in which three additional cells 50, 51 and 52 are provided in parallel. Again these are connected by stop cocks 41 and 42 to enable their isolation for cleaning purposes. In the embodiment of FIG. 4, the cell 30 is omitted and sufficient electrolysis action is available from the cells 50, 51 and 52 to enable the circulating water to be maintained in a superchlorinated state.

A considerable advantage of the above described arrangements that the biocidal agent is continually being recycled and that no added chemicals are required for biocidal activity. The halogens naturally concentrated within the circulating water 4 by the evaporation of the water provide the feed material for the generation of oxyhalite compounds in the electrolysis cell(s) 30, 40, 50, 51 and 52 and these are very effective biocide agents and non-corrosive.

If desired, an additional halogen source in the form of bromine sticks (which are approximately 60% chlorine and 40% bromine) can be placed in the pond 3 and allowed to float on the surface of the water 4. The action of bromine is thought to be particularly effective in breaking up sticky bio-films which breed microbial colonies.

Turning now to FIG. 5, because the demand for free chlorine is dependent largely on the level of particulate pollutants, it is possible to control the current of the cell 30 (or 40 or 50-52) in accordance with the pollution concentration. A particulate sensor 60 is connected to a controllable rectifier 51 which supplies DC current to the cell 30 via an ammeter A, the power being derived from an AC mains supply 62. In the event that increased dust or other particulate pollution is detected, then the DC current to the cell 30 can be increased to ensure a safe level of free chlorine (or oxyhalite) in the circulating water 4, notwithstanding the increased demand.

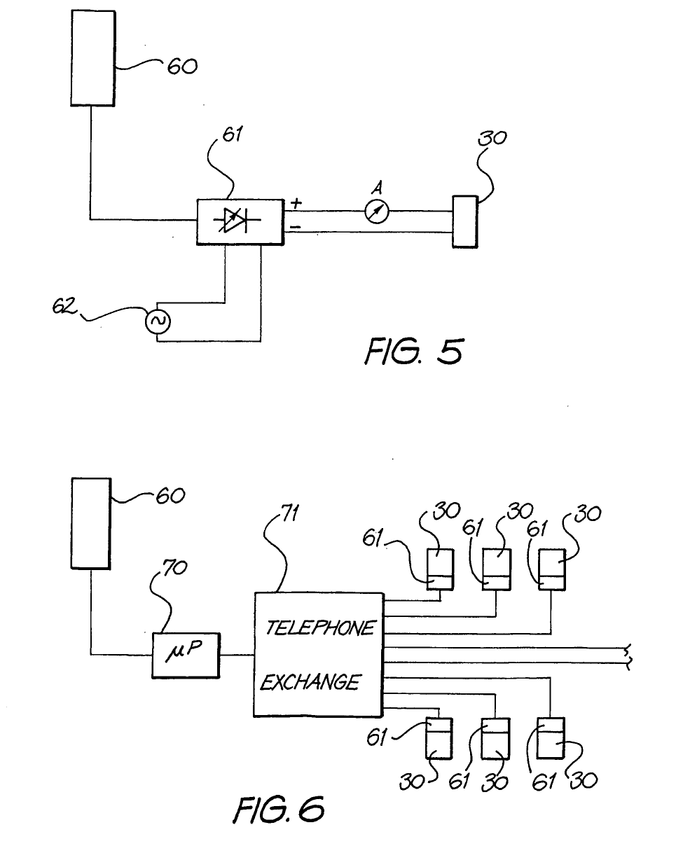

The general principle outlined in FIG. 5 is extended in FIG. 6 where a single sensor 60 is connected via a microprocessor 70 and telephone exchange 71 to control all the electrolytic cells, each with their own controllable rectifier 61, located within a neighbourhood defined by the sensing range of the sensor 60.

### INDUSTRIAL APPLICABILTY

It will be appreciated that the above described arrangements offer a number of very substantial advantages. Firstly, safe biocidal operation is ensured with an adequate buffer provided against days of high pollution levels—which can occur unexpectedly and can be created by building or demolition activity on adjacent premises not merely atmospheric or weather conditions. Secondly a substantial expense in terms of both salaries and chemical costs can be saved in departing from the previous maintenance, shut down and emergency regimes. As the biocide is recycled no chemicals need be purchased and the dangers of handling and transporting chemicals are avoided.

Although the above has been described in relation to cooling towers, it will be appreciated by those skilled in the art that the present invention is equally applicable to scrubbers where the product is effectively sterile air rather than sterile water.

The foregoing describes only some embodiments of the present invention and modification, obvious to those skilled in the art, can be made thereto without departing from the scope of the present invention. For example, one or more electroyltic cells can be placed directly in the pond 3. The electrolysis action itself will guarantee sufficient dispersal of free chlorine throughout the pond 3.

The term “comprising” as used herein is used in the inclusive sense of “having” or “including” and not in the exclusive sense of “consisting only of.

1. A method of operating a cooling tower or like structure having a body of recirculating water, air/water interface means to bring the recirculating water into contact with the air, and biocide means to kill or treat microbial life forms in the recirculating water, said method comprising the step of increasing the activity of said biocide means in response to increases in particulate pollutants in said air.

2. The method as claimed in claim 1 wherein said biocide means comprises an electrolytic cell communicating with said recirculating water and said step of increasing the activity of said cell comprises the step of increasing the current supplied to said cell.

3. The method as claimed in claim 2 including the steps of measuring the level of said particulate pollutants and controlling said cell current in response thereto.

4. The method as claimed in claim 3 wherein a plurality of said cooling towers or like structures are simultaneously controlled by a single device which measures the level of said particulate pollutants.

5. A cooling tower or like structure having a body of recirculating water, air/water interface means to bring the recirculating water into contact with the air, biocide means to kill or treat microbial life forms in the circulating water and means to increase the activity of said biocide means in response to increases in particulate pollutants in said air.

6. The apparatus as claimed in claim 5 wherein said biocide means comprises an electrolytic cell communicating with said recirculating water and said activity increasing means comprises means to increase the current supplied to said cell.

7. The apparatus as claimed in claim 5 including a sensor means to measure the level of said particulate pollutants and connected to said activity increasing means to control same.

8. The apparatus as claimed in claim 7 having a plurality of said cooling towers or like structures each with a corresponding electrolytic cell and activity increasing means, and said sensor means is connected to all said activity increasing means to control same.

9. A cooling tower or like structure in which water is recirculated via a recirculation circuit, the tower or like structure having a pond to retain said water, and pump means to move said water from said pond through a heat source to heat said water and thence to an air/water interface means comprising at least one spray nozzle distributing a spray of water onto fill material in the presence of a counter flow of air whereby said water is cooled by partial evaporation thereof, said air/water interface means being located upstream of said pond, and wherein the recirculation circuit for said water includes at least one electrolytic cell for generating biocide activity wherein no chemicals are added to said water.

10. The cooling tower as claimed in claim 9 wherein said electrolytic cell is located between said heat source and said air/water interface means.

11. The cooling tower as claimed in claim 10 wherein said electrolytic cell is located immediately upstream of said spray nozzle or nozzles.

12. The cooling tower as claimed in claim 9 wherein said electrolytic cell is connected to said pond, is fed from said pond and delivers to said pond.

13. The cooling tower as claimed in claim 9 having first and second electrolytic cells, said first cell being located between said heat source and said air/water interface means and said second cell being connected to said pond, being fed from said pond and delivering to said pond.

14. The cooling tower as claimed in claim 13 wherein said first cell is located immediately upstream of said spray nozzle or nozzles.

15. A method of operating a cooling tower or like structure in which water is recirculated via a recirculation circuit from a pond to retain said water, by pump means which moves said water from said pond through a heat source to heat said water and thence to an air/water interface means having at least one spray nozzle directing a spray of water onto fill material in the presence of a counter flow of air whereby said water is cooled by partial evaporation thereof and after exiting said air/water interface means returns to said pond, said method including the step of including at least one electrolytic cell in the recirculation circuit for said water for generating biocide activity wherein no chemicals are added to said water.

16. The method as claimed in claim 15 including the step of locating said electrolytic cell between said heat source and said air/water interface means.

17. The method as claimed in claim 16 including the step of locating said cell immediately upstream of said spray nozzle or nozzles.

18. The method as claimed in claim 15 including the steps of connecting said cell to said pond, feeding said cell from said pond, and delivering the output of said cell to said pond.

19. The method as claimed in claim 15 including the step of including first and second electrolytic cells in the recirculation circuit for said water, said first cell being located between said heat source and said air/water interface means and said second cell being connected to said pond, being fed from said pond and delivering the output of said second cell to said pond.

20. The method as claimed in claim 19 including the step of locating said first cell immediately upstream of said nozzle or nozzles.

21. A method of operating a cooling tower or like structure having a body of recirculating water, air/water interface means to bring the recirculating water into contact with the air, and biocide means to kill or treat microbial life forms in the recirculating water, said method comprising the step of substantially recycling chemical components of said biocide means.

22. A method as claimed in claim 21 wherein said biocide means comprises at least one electrolytic cell and said method comprising the steps of using said cell to convert halogens present in said water to oxyhalogen compounds, and using said recirculating water to convert said oxyhalogen compounds to said halogens.

23. The method as claimed in claim 22 wherein said halogens include at least one of fluorine, chlorine, bromine and iodine.

24. The method as claimed in claims 22 or 23 wherein said recirculating water contains no added halogen salts.

25. The method as claimed in any one of claims 21-24 including the step of concentrating said chemical components of said biocide means by evaporation of at least some of said recirculating water.

26. A cooling tower or like structure having a body of recirculating water, air/water interface means to bring the recirculating water into contact with the air, biocide means to kill or treat microbial life forms in the recirculating water, and recycling means to recycle chemical components of said biocycide means.

27. The apparatus is claimed in claim 26 wherein said biocide means comprises at least one electrolytic cell communicating with said recirculating water and said recycling means comprises said cell to convert halogens present in said water to oxyhalogen compounds and said recirculating water which converts said oxyhalogen compounds to free halogens.

28. The apparatus as claimed in claim 27 wherein said halogens includes at least one of fluorine, chlorine, bromine and iodine.

29. The apparatus as claimed in claim 27 or 28 wherein said recirculating water contains no added halogen salts.

30. The apparatus as claimed in any one of claims 26-29 wherein said air/water interface means concentrates said chemical components of said biocide means by evaporation of at least some of said recirculating water.

31. A method of reducing corrosion in cooling towers or like structures having a body of recirculating water to which no chemicals are added, air/water interface means to bring the recirculating water into contact with the air, and biocide means to kill or treat microbial life forms in the recirculating water and generate oxyhalogen compounds through the operation thereof, said method comprising the step of converting at least some of oxyhalogen compounds into free halogen.

32. The method as claimed in claim 31 including the step of using an electrolytic cell to convert oxyhalogen compounds into free halogen.

33. The method as claimed in claim 31 or 32 wherein said free halogen includes at least one of fluorine, chlorine, bromine and iodine.

34. The method as claimed in any one of claims 31-33 wherein said recirculating water contains no added halogen salts.

35. The method as claimed in any one of claims 31-34 including the step of concentrating said biocide means by evaporation of at least some of said recirculating water.

36. Apparatus for reducing corrosion in cooling towers or like structures having a body of recirculating water, air/water interface means to bring the recirculating water into contact with the air and biocide means to kill or treat microbial life forms in the recirculating water and generate oxyhalogen compounds through the operation thereof and wherein no chemicals are added to said water, said apparatus comprising converter means to convert at least some of said oxyhalogen compounds into free halogen.

37. The apparatus as claimed in claim 36 wherein said converter means comprises an electrolytic cell in fluid communication with said water.

38. The apparatus as claimed in claim 36 or 37 wherein said free halogen includes at least one of fluorine, chlorine, bromine and iodine.

39. The apparatus as claimed in any one of claims 36-38 wherein said recirculating water contains no added halogen salt.

40. The apparatus as claimed in any one of claims 36-39 wherein said air/water interface means concentrates said biocide means by evaporation of at least some of said recirculating water.

41. A method of maintaining the operation of a cooling tower or like structure substantially free from microbial contamination, said cooling tower comprising a body of recirculating water and air/water interface means to bring the circulating water into contact with the air, said method comprising the step of maintaining said water in a superhalogenated equilibrium state by the continuous production of oxyhalogen compounds.

42. The method as claimed in claim 41 comprising the step of continuously producing said oxyhalogen compounds by electrolysis.

43. The method as claimed in claim 41 or 42 wherein said oxyhalogen compounds include at least one of oxyfluorine, oxychlorine, oxybromine and oxyiodine compounds.

44. The method as claimed in any one of claims 41-43 wherein said recirculating water contains no added halogen salts.

45. The method as claimed in any one of claims 41-44 including the step of concentrating said biocide means by evaporation of at least some of said recirculating water.

46. Apparatus for maintaining the operation of a cooling tower or like structure substantially free from microbial contamination, said cooling tower comprising a body of recirculating water and air/water interface means to bring the recirculating water into contact with the air, said apparatus comprising a biocide means in fluid communication with said water and operable to maintain said water in a superhalogenated equilibrium state by the continuous production of oxyhalogen compounds.

47. The apparatus as claimed in claim 46 wherein said biocide means comprises at least one electrolytic cell.

48. The apparatus as claimed in claims 46 or 47 wherein said oxyhalogen compounds includes at least one of oxyfluorine, oxychlorine, oxybromine and oxyiodine compounds.

49. The apparatus as claimed in any one of claims 46-48 wherein said recirculating water contains no added halogen salts.

50. The apparatus as claimed in any one of claims 46-49 wherein said air/water interface means concentrates said biocide means by evaporation of at least some of said recirculating water.

51. A method of closing down the operation of a cooling tower or like structure to permit resumption of said operation within a short period, said cooling tower comprising a body of recirculating water, air/water interface means to bring the circulating water into contact with the air, and biocide means to kill or treat microbial life forms in the recirculating water, said method comprising the steps of achieving a equilibrium state of superhalogenation in said circulating water prior to closing down, and maintaining said equilibrium state of superhalogen in said water after shut down, said state of superhalogenation being achieved and maintained by the production of oxyhalogen compounds from said water.

52. The method as claimed in claim 51 including the step of continuously producing said oxyhalogen compounds.

53. The method as claimed in claim 51 or 52 including the step of producing said oxyhalogen compounds by electrolysis of said water.

54. The method as claimed in any one of claims 51-53 wherein said oxyhalogen compounds include at least one of oxyfluorine, oxychlorine, oxybromine and oxyiodine compounds.

55. The method as claimed in any one of claims 51-54 wherein said recirculating water contains no added halogen salts.

56. The method as claimed in any one of claims 51-55 including the step of concentrating said biocide means by evaporation of at least some of said recirculating water.

57. Apparatus for closing down the operation of a cooling tower or like structure to permit resumption of said operation within a short period, said cooling tower comprising a body of recirculating water, air/water interface means to bring the recirculating water into contact with the air, and biocide means to kill or treat microbial life forms in the recirculating water, said apparatus comprising first halogenation means to achieve a equilibrium state of superhalogenation in said water prior to closing down, and second halogenation means to maintain said equilibrium state of superhalogenation in said water after shut down, said first and second halogenation means being operable to produce oxyhalogen compounds from said water.

58. The apparatus as claimed in claim 57 wherein said first halogenation means comprises at least one electrolytic cell.

59. The apparatus as claimed in claim 57 or 58 wherein said second halogenation means comprises at least one electrolytic cell.

60. The apparatus as claimed in claim 57 wherein said first halogenation means comprises at least one electrolytic cell and said second halogenation means comprises at least one electrolytic cell.

61. The apparatus as claimed in claim 60 wherein said first halogenation means and said second halogenation means comprise the same electrolytic cell or cells.

62. The apparatus as claimed in any one of claims 57-61 wherein said oxyhalogen compounds includes at least one of oxyfluorine, oxychlorine, oxybromine and oxyiodine compounds.

63. A method of increasing the sterility of gas by bringing same into contact with superhalogenated water, said method comprising the step of superhalogenating said water by electrolysis.

64. The method as claimed in claim 63 wherein said gas is air.

65. The method as claimed in claim 63 or 64 wherein said gas is brought into contact with droplets of said water.

66. The method as claimed in claim 65 wherein said gas and said water droplets flow in opposite directions.

67. Apparatus for increasing the sterility of gas, said apparatus comprising first means to bring said gas into contact with water, and electrolysis means to superhalogenate said water.

68. The apparatus as claimed in claim 67 wherein said gas is air.

69. The apparatus as claimed in claim 67 or 68 wherein said first means includes a nozzle means to create droplets of said water.

70. The apparatus as claimed in claim 69 wherein said droplets and said gas move in opposite directions.
